(19) United States
(12) Patent Application Publication
(10) Pub. No.: US 2013/0123541 A1
(43) Pub. Date: May 16, 2013

(54) METHOD OF PREPARING 3,3,5,5-TETRAMETHYLCYCLOHEXANONE

(71) Applicants: Herbert Koller, Wien (AU); Michael Pyerin, Brunn Am Gebirge (AT); Federico Sbrogio, Montecchio Maggiore (IT)

(72) Inventors: Herbert Koller, Wien (AU); Michael Pyerin, Brunn Am Gebirge (AT); Federico Sbrogio, Montecchio Maggiore (IT)

(73) Assignee: MERZ PHARMA GmbH & CO. KGaA, Frankfurt am main (DE)

(21) Appl. No.: 13/378,773

(22) PCT Filed: Jun. 28, 2010
(86) PCT No.: PCT/EP2010/003923

§ 371 Date: Jan. 31, 2012

(30) Foreign Application Priority Data
Jun. 29, 2009 (EP) EP 09008464.1

(51) Int. Cl. C07C 45/69; C07C 209/62

(52) U.S. Cl. CPC C07C 45/69; C07C 209/62
USPC 564/462; 568/350

(57) ABSTRACT

Method of preparing 3,3,5,5-tetramethylcyclohexanone comprising step (i): (i) converting isophorone to 3,3,5,5-tetramethylcyclohexanone in the presence of methylmagnesium chloride. The thus prepared 3,3,5,5-tetramethylcyclohexanone may be employed in a method of preparing 1-amino-1,3,3,5,5-pentamethylcyclohexane (Neramexane) or a pharmaceutically acceptable salt thereof.

### FIELD OF THE INVENTION

The present invention relates to a method of preparing 3,3,5,5-tetramethylcyclohexanone. Said product may be used as an intermediate in the manufacture of 1-amino-1,3,3,5,5-pentamethylcyclohexane (Neramexane) or a pharmaceutically acceptable salt thereof.

### BACKGROUND OF THE INVENTION

1-amino-1,3,3,5,5-pentamethylcyclohexane (Neramexane) and pharmaceutically acceptable salts thereof are valuable agents for the continuous therapy of patients suffering from diseases and conditions such as tinnitus, and nystagmus.

Methods of preparing these agents are already known.

In one method, commercially available isophorone is converted to Neramexane in a reaction sequence comprising five steps according to the following reaction scheme (W. Danysz et al., Current Pharmaceutical Design, 2002, 8, 835-843):

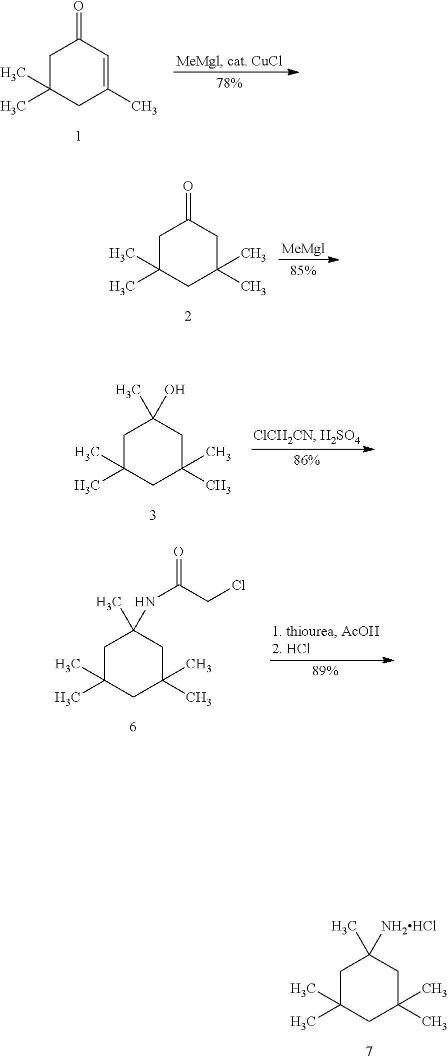

In the first step of the sequence, isophorone 1 is converted to 3,3,5,5-tetramethylcyclohexanone 2 by CuCl-catalyzed conjugate addition of methyl-magnesium iodide. The yield of target compound is 78% by weight.

It is also known that methylmagnesium bromide may be added to isophorone in the presence of cuprous chloride to result in 3,3,5,5-tetramethylcyclohexanone in a yield of 82.5% by weight. As by-product, 1,3,5,5-tetramethylcyclohexadiene in a yield of 6.9% by weight was obtained (Kharasch et al., J. Am. Cem. Soc., 1941, 63, 2308).

The same publication discloses in its experimental part (page 2313) the addition of methylmagnesium chloride to isophorone in the presence of ferric chloride. However, no 3,3,5,5-tetramethylcyclohexanone has been formed, but products completely different therefrom have been isolated.

In the second step, 3,3,5,5-tetramethylcyclohexanone 2 is converted to 1,3,3,5,5-pentamethylcyclohexanol 3 by using methylmagnesium iodide.

In the third step of the sequence, said cyclohexanol 3 is converted to 1-chloroacetamido-1,3,3,5,5-pentamethylcyclohexane 6 by chloroacetonitrile in a Ritter reaction.

In the subsequent fourth step, cleavage of the chloroacetamido group in amide 6 with thiourea in acetic acid, and acidification of the resulting amine with hydrochloric acid in the final fifth step of the reaction sequence results in Neramexane (1-amino-1,3,3,5,5-pentamethylcyclohexane) 7 in the form of its hydrochloride.

### OBJECTS OF THE INVENTION

One object of the invention is to improve one or more of the individual reaction steps of the above referenced reaction sequence in order to provide a method of preparing 1-amino-1,3,3,5,5-pentamethylcyclohexane or a pharmaceutically acceptable salt thereof that allows an advantageous realization on an economical industrial scale. It is in another object to minimize the amount of waste and/or unused chemicals produced during the manufacture of Neramexane or a pharmaceutically acceptable salt thereof. It is a further object to optimize or improve the yield and/or selectivity and/or product quality in regard to Neramexane or a pharmaceutically acceptable salt thereof. Particularly, the subject application aims to improve above step (i), i.e. reaction of isophorone with a methylmagnesium halide. Such an improved method may be regarded as one prerequisite for an advantageous manufacture of Neramexane or a pharmaceutically acceptable salt thereof on an economical industrial scale.

### SUMMARY OF THE INVENTION

The present invention relates to an improved synthesis of 3,3,5,5-tetramethylcyclohexanone. Said compound is an intermediate in the production of 1-amino-1,3,3,5,5-pentamethylcyclohexane (Neramexane) or a pharmaceutically acceptable salt thereof.

Specifically, the present invention relates to a method of preparing 3,3,5,5-tetramethylcyclohexanone comprising step (i):
(i) converting isophorone to 3,3,5,5-tetramethylcyclohexanone in the presence of methylmagnesium chloride.

In one embodiment, step (i) is performed in the presence of a copper compound.

In one embodiment, the copper compound is a copper(I) halide.

In one embodiment, the copper(I) halide is copper(I) iodide.

In one embodiment, step (i) is performed in the presence of a lithium compound.

In one embodiment, the lithium compound is a lithium halide.

In one embodiment, the lithium halide is lithium chloride.

In one embodiment, the molar ratio of copper(I) halide to lithium halide is in the range of from 1:1.5 to 1:2.5.

In one embodiment, step (i) is performed in a solvent comprising an ether.

In one embodiment, the ether is tetrahydrofurane.

In one embodiment, isophorone is converted to 3,3,5,5-tetramethylcyclohexanone by using methylmagnesium chloride, copper(I) iodide and lithium chloride in tetrahydrofurane.

In one embodiment, a solution comprising methylmagnesium chloride in tetrahydrofurane is added to a solution comprising isophorone, copper(I) iodide and lithium chloride.

The invention also relates to the use of methylmagnesium chloride for converting isophorone to 3,3,5,5-tetramethylcyclohexanone.

In one embodiment of the use, methylmagnesium chloride is dissolved in tetrahydrofurane.

In another aspect, the invention relates to a method of preparing 1-amino-1,3,3,5,5-pentamethylcyclohexane or a pharmaceutically acceptable salt thereof, such as the hydrochloride or the mesylate thereof, comprising step (i):
(i) converting isophorone to 3,3,5,5-tetramethylcyclohexanone by using methylmagnesium chloride.

In one embodiment, said methylmagnesium chloride is free of ethylmagnesium chloride.

In one aspect, the invention relates to 1-amino-1,3,3,5,5-pentamethylcyclohexane or a pharmaceutically acceptable salt thereof which is substantially free of 1-amino-3-ethyl-1,3,5,5-tetramethylcyclohexane and, optionally, free of 1-amino-1-ethyl-3,3,5,5-tetramethylcylohexane; or a pharmaceutically acceptable salt thereof.

It has unexpectedly been discovered that the method according to the invention shortens the reaction time as compared to the reaction time as disclosed in the methods of the prior art resulting in high yields of the target compound. Moreover, the formation of by-products such as 1,3,5,5-tetramethylcyclohexadiene in step (i) is suppressed as far as possible, therefore also avoiding complex distillation processes for the purification of 3,3,5,5-tetramethylcyclohexanone.

The novel method of preparing 3,3,5,5-tetramethylcyclohexanone from isophorone improves the hitherto known method of producing Neramexane as referenced in the Background section of this application. It may be advantageously performed on an economical industrial scale.

### DETAILED DESCRIPTION OF THE INVENTION

This invention relates to a method of preparing 3,3,5,5-tetramethylcyclohexanone starting from isophorone.

Specifically, this invention relates to a method of preparing 3,3,5,5-tetramethylcyclohexanone comprising step (i):
(i) converting isophorone to 3,3,5,5-tetramethylcyclohexanone in the presence of methylmagnesium chloride.

Methylmagnesium chloride is a Grignard reagent. It may be produced by reacting magnesium with methyl chloride.

In said conversion of step (i), said methylmagnesium chloride adds to isophorone in a conjugated 1,4-addition. Accordingly, after working up the reaction mixture that had previously comprised isophorone and methylmagnesium chloride, 3,3,5,5-tetramethylcyclohexanone is obtained.

In one embodiment, a catalyst is added to benefit the 1,4-addition of the Grignard reagent over the possible 1,2-addition.

In one embodiment, step (i) is performed in the presence of a copper compound.

In one embodiment, the copper compound is a copper(I) compound.

In one embodiment, the copper(I) compound is a copper(I) halide.

Accordingly, the copper(I) halide is selected from the fluoride, chloride, bromide or iodide.

In one embodiment, the copper(I) halide is copper(I) chloride or copper(I) iodide.

In one embodiment, the copper(I) halide is copper(I) iodide.

It has been discovered that the selectivity of the reaction may be further improved by performing step (i) not only in the presence of a copper compound such as a copper(i) halide such as copper(I) chloride or copper(I) iodide, but also in the presence of a lithium compound.

In one embodiment, the lithium compound is a lithium halide.

Accordingly, said lithium halide is selected from lithium fluoride, lithium chloride, lithium bromide, lithium iodide.

In one embodiment, said lithium halide is lithium chloride.

In one embodiment, the molar ratio of copper(I) halide to lithium halide is in the range of from 1:1.5 to 1:2.5.

In one embodiment, the molar ratio of copper(I) chloride or copper(I) iodide to lithium chloride is in the range of from 1:1.5 to 1:2.5.

In one embodiment, the molar ratio of copper(I) iodide to lithium chloride is in the range of from 1:1.5 to 1:2.5.

In one embodiment, said ratio is about 1:2, or is 1:2.

The reaction according to step (i) commonly is performed in a solvent.

In one embodiment, said solvent comprises an ether, or the solvent is an ether.

Ethers may be selected from diethyl ether, 1,4-dioxane, or tetrahydrofurane.

In one embodiment, said ether is tetrahydrofurane.

In one embodiment, isophorone is converted to 3,3,5,5-tetramethylcyclohexanone by using methylmagnesium chloride, copper(I) chloride or copper (I) iodide and lithium chloride in tetrahydrofurane.

In one embodiment, isophorone is converted to 3,3,5,5-tetramethylcyclohexanone by using methylmagnesium chloride, copper (I) iodide and lithium chloride in tetrahydrofurane.

In one embodiment, isophorone, the copper compound such as copper (I) halide (e.g. copper(I) iodide or copper(I) chloride) and, optionally, the lithium compound such as lithium halide (e.g. lithium chloride), are provided in a solvent, and the Grignard reagent, optionally dissolved in a solvent, is added to said mixture.

In one embodiment, methylmagnesium chloride is dissolved in tetrahydrofurane.

In one embodiment, the concentration of methylmagnesium chloride in tetrahydrofurane is from 15 to 30% by weight, or 20 to 25% by weight based on the total amount of methylmagnesium chloride and tetrahydrofurane.

In one embodiment, the concentration of methylmagnesium chloride in tetrahydrofurane is 23% by weight based on the total amount of methylmagnesium chloride and tetrahydrofurane.

In one embodiment, more than one molar equivalent methylmagnesium chloride is employed per one molar equivalent isophorone.

In one embodiment, from 1.0 to 1.75 molar equivalents methylmagnesium chloride, or from 1.2 to 1.5 molar equivalents methylmagnesium chloride are employed per one molar equivalent isophorone.

In one embodiment, the concentration of methylmagnesium chloride in tetrahydrofurane is 23% by weight based on the total amount of methylmagnesium chloride and tetrahydrofurane, and 10% by weight catalyst (one molar equivalent copper(I) iodide and two molar equivalents lithium chloride) based on the amount of methylmagnesium chloride and tetrahydrofurane are employed.

In one embodiment, from 0.1 to 0.25 molar equivalents lithium chloride and from 0.05 to 0.125 molar equivalents copper(I) iodide per one molar equivalent isophorone are employed.

In another embodiment, methylmagnesium chloride is reacted with the copper compound such as a copper(I) halide (e.g. copper (I) iodide or copper(I) chloride), optionally in the presence of a lithium compound such as lithium halide (e.g. lithium chloride). In one embodiment, said mixture is added to isophorone. In another embodiment, isophorone is added to said mixture.

In another embodiment, a mixture of isophorone, copper (I) iodide and lithium chloride is provided in tetrahydrofurane. Methylmagnesium chloride which was previously dissolved in tetrahydrofurane respectively prepared in tetrahydrofurane, is added to said mixture.

In one embodiment, the above-defined embodiments are performed such that the temperature can be controlled.

In one embodiment, the addition is performed such that the temperature may be kept in a relatively narrow temperature range.

In one embodiment, the conversion in step (i) is performed at a temperature of from −5° C. to 20° C., or 0° C. to 20° C., or −5° C. to 15° C., or −1° C. to 10° C.

The addition of the Grignard reagent to isophorone commonly proceeds rather fast. Usually, the reaction may be terminated after three hours or two hours or even one hour, depending on the reaction temperature employed.

After the reaction of isophorone with the Grignard reagent, the reaction mixture may be treated with water in order to destroy an excess of Grignard reagent, if any employed, respectively to destroy basic magnesium compounds.

In one embodiment, an acid such as hydrochloric acid, or an ammonium salt is added to support the formation of 3,3,5,5-tetramethylcyclohexanone.

In one embodiment, the 3,3,5,5-tetramethylcyclohexanone formed in step (i) isolated by extracting the aqueous mixture with an appropriate organic solvent such as methylene chloride or toluene or petroleum ether. Subsequent to extracting, the solvent may be removed by distillation.

In one embodiment, the residue comprising crude 3,3,5,5-tetramethylcyclohexanone as obtained and isolated may be employed without purification in step (ii) of the reaction sequence.

In another embodiment, subsequent to extracting, the extract may be dried according to known methods. For example, the extract may be dried over sodium sulphate. After separating off said sulphate by filtration, the solvent may be removed by distillation. The residue comprising crude 3,3,5,5-tetramethylcyclohexanone as obtained and isolated may be employed without purification in step (ii) of the reaction sequence.

In one embodiment, the yield of crude 3,3,5,5-tetramethylcyclohexanone as obtained and isolated in step (i) is in the range of from 88% to 96% by weight.

In one embodiment, the crude 3,3,5,5-tetramethylcyclohexanone contains the target compound in an amount of at least 93% by weight as can be determined by gas-liquid chromatography. The amount of the above addressed by-products commonly is less than 1% by weight.

In one embodiment, the crude 3,3,5,5-tetramethylcyclohexanone is distilled in order to further purify it.

In another embodiment, the crude 3,3,5,5-tetramethylcyclohexanone is employed in step (ii) of the sequence as addressed above, i.e. 3,3,5,5-tetramethylcyclohexanone is not subjected to a purification step.

In one embodiment, the crude 3,3,5,5-tetramethylcyclohexanone that is a liquid at ambient temperature (25° C.) is not subjected to a purification step of distillation or chromatography.

The direct use of the crude product in subsequent step (ii) is possible, since the reaction of isophorone with methylmagnesium chloride, contrary to the reaction with methylmagnesium iodide or methyl magnesium bromide, suppresses the formation of the above addressed by-product as far as possible.

In summary, the use of methylmagnesium chloride for converting isophorone to 3,3,5,5-tetramethylcyclohexanone is advantageous over the respective uses of methylmagnesium bromide and methylmagnesium iodide. This particularly concerns the suppressing of by-products and/or the achievable high yields and/or the possibility of applying the obtained compound as crude product in step (ii) of the reaction sequence as addressed in the Background section. This is particularly advantageous in view of an industrial realization.

Accordingly, the present invention also relates to the use of methylmagnesium chloride for converting isophorone to 3,3,5,5-tetramethylcyclohexanone.

In one embodiment of the use, methylmagnesium chloride is dissolved in tetrahydrofurane.

In one embodiment, the method of preparing 3,3,5,5-tetramethylcyclohexanone may be performed in a method for preparing 1-amino-1,3,3,5,5-pentamethylcyclohexane or a pharmaceutically acceptable salt thereof as addressed in the Background section of this application.

Accordingly, in one aspect, the invention also relates to a method of preparing 1-amino-1,3,3,5,5-pentamethylcyclohexane or a pharmaceutically acceptable salt thereof, comprising step (i):
(i) converting isophorone to 3,3,5,5-tetramethylcyclohexanone in the presence of methylmagnesium chloride.

For the purpose of this disclosure, the term “pharmaceutically acceptable salts” refers to salts of neramexane that are physiologically tolerable and do not typically produce untoward reactions when administered to a mammal (e.g., human). Typically, the term “pharmaceutically acceptable salt” means approved by a regulatory agency of the Federal or a state government or listed in the U.S. Pharmacopeia or other generally recognized pharmacopeia for use in mammals, and more particularly in humans.

Conversion of 1-amino-1,3,3,5,5-pentamethylcyclohexane to a pharmaceutically acceptable salt thereof is accomplished in conventional fashion by admixture of the base with at least one molecular equivalent of a selected acid in an inert organic solvent. Isolation of the salt is carried out by techniques known to the art such as inducing precipitation with a non-polar solvent (e.g. ether) in which the salt has limited solubility. The nature of the salt is not critical, provided that it is non-toxic and does not substantially interfere with the desired pharmacological activity.

Examples of pharmaceutically acceptable salts are those formed with hydrochloric, hydrobromic, methanesulfonic, acetic, succinic, maleic, citric acid, and related acids.

Further pharmaceutically acceptable salts include, but are not limited to, acid addition salts, such as those made with hydroiodic, perchloric, sulfuric, nitric, phosphoric, propionic, glycolic, lactic, pyruvic, malonic, fumaric, tartaric, benzoic, carbonic, cinnamic, mandelic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid.

The conversion in step (i) may be performed according to any one of the embodiments as disclosed above.

In one embodiment, besides 1-amino-1,3,3,5,5-pentamethylcyclohexane, respectively a salt thereof, further amino compounds may be formed and detected, which are different from the target compound 1-amino-1,3,3,5,5-pentamethylcyclohexylamine or the respective salt thereof.

In one embodiment, 1-amino-3-ethyl-1,3,5,5-tetramethylcyclohexane may be formed as a side-product. It may be detected e.g. by gas chromatographical analysis. Since this compound has two chiral centers, two diastereomers may be detected.

In one embodiment, the occurrence of 1-amino-3-ethyl-1,3,5,5-tetramethylcyclohexane may be attributed to the addition of an ethyl group instead of a methyl group to isophorone in step (i) to yield the respective 3-ethyl-3,5,5-trimethylcyclohexanone. If the sequence as described in the Background section is performed, i.e. conversion to the hydroxyl compound, subsequent conversion to the Ritter product and subsequent conversion to neramexane via the reaction with e.g. thiourea, said amine respectively a salt thereof is formed.

In one embodiment, the occurrence of said side-products may be attributed to the contamination of the employed methylmagnesium chloride with ethylmagnesium chloride.

In one embodiment, the occurrence of said undesired side-products may be suppressed or reduced by employing a purified methylmagnesium chloride in step (i) which is free of ethylmagnesium chloride.

In one embodiment, methylmagnesium chloride contains less than 1.0% by weight ethylmagnesium chloride based on the total amount of methylmagnesium chloride and ethylmagnesium chloride, or less than 0.5% by weight, or less than 0.1% by weight.

In another embodiment, 3-ethyl-3,5,5-trimethylcyclohexanone formed in step (i) may be removed from 3,3,5,5-tetramethylcyclohexane by distillation.

In another embodiment, it is not necessary to purify 3,3,5,5-tetramethylcyclohexane formed in step (i). In one embodiment, a purification step is performed at a later stage, e.g. at the stage of neramexane formation or formation of a salt thereof.

In one embodiment, said side-products may be removed from neramexane by purifying the amine. In one embodiment, the amine may be purified by distillation wherein said side-products are removed.

In another embodiment, the salt obtained from neramexane is purified. In one embodiment, said salt may be purified by a step of re-crystallization. A suitable solvent is e.g. a solvent selected from the solvents as used for the salt formation. In one embodiment, the solvent is anisole. In one embodiment, the salt is the mesylate.

In another embodiment, 1-amino-1-ethyl-3,3,5,5-tetramethylcyclohexane or a salt thereof may be additionally detected provided 3,3,5,5-tetramethylcyclohexanone is converted to the respective hydroxyl compound by employing a Grignard reagent as referenced in the Background section. In one embodiment, methylmagnesium chloride is employed.

In one embodiment, the occurrence of 1-amino-1-ethyl-3,3,5,5-tetramethylcyclohexane may be attributed to the addition of an ethyl group instead of a methyl group to the carbonyl group of 3,3,5,5-tetramethylcyclohexanone. If the sequence as described in the Background section is performed, i.e. conversion to the Ritter product and subsequent conversion to neramexane, said amine respectively a salt thereof is formed.

The formation of said amine may be suppressed or prevented, respectively the removal of said compound may be performed by the methods as described above in connection with 1-amino-3-ethyl-1,3,5,5-tetramethylcyclohexane.

Accordingly, in one aspect, the invention relates to 1-amino-1,3,3,5,5-pentamethylcyclohexane or a pharmaceutically acceptable salt thereof which is substantially free of 1-amino-3-ethyl-1,3,5,5-tetramethylcyclohexane and, optionally, free of 1-amino-1-ethyl-3,3,5,5-tetramethylcylohexane; or a pharmaceutically acceptable salt thereof.

The term “substantially free of” defines an amount of less than 0.5% by weight of said side-products based on the total amount of 1-amino-1,3,3,5,5-pentamethylcyclohexane or a pharmaceutically acceptable salt thereof and said side-products.

### EXAMPLE

A mixture of 93 g methylmagnesium chloride and 372 g tetrahydrofurane is added by dropping to a stirred mixture of 139 g isophorone, 19 g copper(I) iodide, 8.4 g lithium chloride and 1,550 g tetrahydrofurane, wherein the inorganic compounds have been dissolved by stirring prior to the dropping. The dropping rate is selected such that the temperature of the mixture is maintained between 5 and 15° C. After the addition is terminated, the mixture is stirred for another 60 minutes. Subsequently, diluted hydrochloric acid is added to decompose an excess of methylmagnesium chloride, and to decompose basic magnesium compounds. The mixture is extracted twice with petroleum ether. The extracts are combined and washed with ammonia. Subsequently, the solvent is distilled off. The crude yield of target compound is quantitative (153 g). The content of 3,3,5,5-tetramethylcyclohexanone in the crude product is about 91% by weight as determined by gas-liquid chromatography. The crude product contained approximately 2% by weight non-reacted isophorone, less than 1% by weight 1,3,5,5-tetramethylcyclohexanol or olefins generated from said compound, and 1 by weight 1-hydroxy-1,3,3,5,5-pentamethylcyclohexane.

1.-16. (canceled)

17. A method of preparing 3,3,5,5-tetramethylcyclohexanone comprising step (i):
(i) converting isophorone to 3,3,5,5-tetramethylcyclohexanone in the presence of methylmagnesium chloride.

18. The method according to claim 17, wherein step (i) is performed in the presence of a copper compound.

19. The method according to claim 18, wherein the copper compound is a copper(I) halide.

20. The method according to claim 19, wherein the copper(I) halide is copper(I) iodide.

21. The method according to claim 17, wherein step (i) performed in the presence of a lithium compound.

22. The method according to claim 21, wherein the lithium compound is a lithium halide,

23. The method according to claim 22, wherein the lithium halide is lithium chloride.

24. The method according to claim 22, wherein the molar ratio of copper(I) halide to lithium halide is in the range of from 1:1.5 to 1:2.5.

25. The method according to claim 17, wherein step (i) is performed in a solvent comprising an ether.

26. The method according to claim 25, wherein the ether is tetrahydrofuran.

27. The method according to claim 17, wherein in step (i) isophorone is converted to 3,3,5,5-tetramethylcyclohexanone in the presence of methylmagnesium chloride, copper(I) iodide and lithium chloride in tetrahydrofuran.

28. The method according to claim 27, wherein a solution comprising methylmagnesium chloride in tetrahydrofuran is added to a solution comprising isophorone, copper(I) iodide and lithium chloride.

29. A method of preparing 1-amino-1,3,3,5,5-pentamethylcyclohexane or a pharmaceutically acceptable salt thereof, comprising step (i):
(i) converting isophorone to 3,3,5,5-tetramethylcyclohexanone in the presence of methylmagnesium chloride.

30. The method according to claim 29, wherein step (i) is performed in the presence of a copper compound.

31. The method according to claim 17, wherein the methylmagnesium chloride is free of ethylmagnesium chloride.

32. 1-Amino-1,3,3,5,5-pentamethylcyclohexane or a pharmaceutically acceptable salt thereof which is substantially free of 1-amino-3-ethyl-1,3,5,5-tetramethylcyclohexane and, optionally, free of 1-amino-1-ethyl-3,3,5,5-tetramethylcylohexane; or a pharmaceutically acceptable salt thereof.

33. The method according to claim 30, wherein the copper compound is a copper(I) halide.

34. The method according to claim 33, wherein the copper(I) halide is copper(i) iodide.

35. The method according to claim 29, wherein step (i) is performed in the presence of a lithium compound.

36. The method according to claim 35, wherein the lithium compound is a lithium halide.

37. The method according to claim 36, wherein the lithium halide is lithium chloride.

38. The method according to claim 35, wherein the molar ratio of copper(I) halide to lithium halide is in the range of from 1:1.5 to 1:2.5.

39. The method according to claim 29, wherein step (i) is performed in a solvent comprising an ether.

40. The method according to claim 39, wherein the ether is tetrahydrofuran.

41. The method according to claim 29, wherein in step (i) isophorone is converted to 3,3,5,5-tetramethylcyclohexanone in the presence of methylmagnesium chloride, copper(I) iodide and lithium chloride in tetrahydrofuran.

42. The method according to claim 41, wherein a solution comprising methylmagnesium chloride in tetrahydrofuran is added to a solution comprising isophorone, copper(I) iodide and lithium chloride.

43. The method according to claim 29, wherein the methylmagnesium chloride is free of ethylmagnesium chloride.
